(19) United States
(12) Patent Application Publication
(10) Pub. No.: US 2024/0091027 A1
(43) Pub. Date: Mar. 21, 2024

(54) IMPACTION HANDLE FOR IMPLANTING A TIBIAL TRAY OF A FIXED-BEARING ORTHOPAEDIC KNEE PROSTHESIS AND ASSOCIATED METHOD OF MAKING THE SAME

(71) Applicant: DEPUY IRELAND UNLIMITED COMPANY, RINGASKIDDY (IE)

(72) Inventors: Cole T. Brown, Winona Lake, IN (US); Jason M. Chavarria, Warsaw, IN (US); Jenna L. Faas, Warsaw, IN (US); Colin M. Lank, Warsaw, IN (US); Cory A. Shulaw, Warsaw, IN (US); Christel M. Wagner, Warsaw, IN (US); Alasdair J.J. Mercer, Leeds (GB); Benjamin R. Powers, Colombia City, IN (US)

(21) Appl. No.: 17/946,497

(22) Filed: Sep. 16, 2022

(51) Int. Cl. A61F 2/46; A61F 2/38

(52) U.S. Cl. CPC A61F 2/461; A61F 2/389; A61B 2017/0046

(57) ABSTRACT

An impaction handle for use during a surgical procedure to implant a fixed-bearing tibial tray into a surgically-prepared proximal end of a tibia is disclosed. The impaction handle includes an impact plate defining a proximal end of the impaction handle and an impact head defining a distal end of the impaction handle. The impaction handle also has an elongated shaft extending between the impact plate and the impact head, along with a locking mechanism to lock the handle to the tibial tray. The locking mechanism includes a thumbwheel positioned in the impact head and a locking shaft secured to the thumbwheel.

### TECHNICAL FIELD

The present disclosure relates generally to orthopaedic surgical instruments and, more particularly, to surgical instruments used to install a fixed-bearing tibial tray of an orthopaedic knee prosthesis.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. A typical knee prosthesis includes a tibial tray, a femoral component, and a polymer insert or bearing positioned between the tibial tray and the femoral component.

To facilitate the replacement of the natural joint with a prosthesis, orthopaedic surgeons use a variety of orthopaedic surgical instruments such as, for example, impaction handles, cutting blocks, drill guides, and other surgical instruments.

### SUMMARY

According to one aspect, an orthopaedic surgical instrument for use during a surgical procedure to implant a tibial tray into a surgically-prepared proximal end of a tibia includes an impaction handle. The impaction handle includes an impact plate defining a proximal end of the impaction handle and an impact head defining a distal end of the impaction handle. The impact head has an impact surface that is sized and shaped to be positioned on a superior surface of the tibial tray when the impaction handle is used to impact the tibial tray. An elongated body extends between the impact plate and the impact head. A locking mechanism is positioned in the impact head. The locking mechanism includes a thumbwheel that is rotatable relative to the impact head and a locking shaft that is secured to a distal surface of the thumbwheel such that a threaded distal end of the locking shaft extends outwardly through an opening formed in the impact surface of the impact head. The threaded distal end of the locking shaft is configured to be threadingly received into a threaded bore formed in the tibial tray. Rotation of the thumbwheel causes rotation of the locking shaft so as to cause its threaded distal end to selectively threadingly engage the threaded bore of the tibial tray when the threaded distal end of the locking shaft is positioned therein. The impact plate, the impact head, and the elongated shaft of the impaction handle collectively define a single monolithic polymer component.

In an embodiment, rotation of the thumbwheel in a first direction causes the threaded distal end of the locking shaft to threadingly engage the threaded bore of the tibial tray when the threaded distal end of the locking shaft is positioned therein. In such an embodiment, rotation of the thumbwheel in a direction opposite the first direction causes the threaded distal end of the locking shaft to threadingly disengage the threaded bore of the tibial tray so as to allow the locking shaft to be removed therefrom.

In another embodiment, the locking mechanism further includes a spring with such a spring asserting a spring bias on the distal surface of the thumbwheel so as to urge the thumbwheel toward the impact plate.

In an embodiment, the impact plate, the impact head, and the elongated shaft of the impaction handle are constructed of polyphenylsulfone.

According to another aspect, an orthopaedic surgical instrument for use during a surgical procedure to implant a tibial tray into a surgically-prepared proximal end of a tibia includes a polymer impaction insert and a metallic impaction handle removably secured to the impaction insert. The impaction insert includes a proximal surface and a distal surface that defines an impact surface that is sized and shaped to be positioned on a superior surface of the tibial tray when the impaction insert is used to impact the tibial tray. The impaction insert also has a central bore extending between the proximal surface and the distal surface of the impaction insert. The impaction handle includes an impact plate defining a proximal end of the impaction handle and an impact head defining a distal end of the impaction handle. The impact head has an impact surface that is sized and shaped to be positioned on the proximal surface of the impaction insert. An elongated body extends between the impact plate and the impact head. A locking mechanism is positioned in the impact head. The locking mechanism includes a thumbwheel that is rotatable relative to the impact head and a locking shaft secured to a distal surface of the thumbwheel such that a threaded distal end of the locking shaft extends outwardly through an opening formed in the impact surface of the impact head and through the central bore of the impaction insert. The threaded distal end of the locking shaft is configured to be threadingly received into a threaded bore formed in the tibial tray. Rotation of the thumbwheel causes rotation of the locking shaft so as to cause its threaded distal end to selectively threadingly engage the threaded bore of the tibial tray when the threaded distal end of the locking shaft is positioned therein.

In an embodiment, rotation of the thumbwheel in a first direction causes the threaded distal end of the locking shaft to threadingly engage the threaded bore of the tibial tray when the threaded distal end of the locking shaft is positioned therein. In such an embodiment, rotation of the thumbwheel in a direction opposite the first direction causes the threaded distal end of the locking shaft to threadingly disengage the threaded bore of the tibial tray so as to allow the locking shaft to be removed therefrom.

In another embodiment, the locking mechanism further includes a spring with such a spring asserting a spring bias on the distal surface of the thumbwheel so as to urge the thumbwheel toward the impact plate.

In an embodiment, the impaction insert is constructed of polyphenylsulfone, and the impaction handle is constructed of stainless steel.

In another embodiment, the impact surface of the impact head of the impaction handle has a medial buttress and a lateral buttress formed therein. In such an embodiment, the proximal surface of the impaction insert includes a medial recess that is sized and shaped to receive the medial buttress of the impact surface of the impact head of the impaction handle and a lateral recess that is sized and shaped to receive the lateral buttress of the impact surface of the impact head of the impaction handle.

In an embodiment, the locking shaft of the impaction handle has an annular ring formed thereon and the impaction insert has an annular groove defined therein. The annular groove is coaxial with, and opens into, the central bore of the impaction insert. The annular ring of the locking shaft is captured in the annular groove so as to secure the impaction insert to the impaction handle.

According to another aspect, an orthopaedic system for use in a surgical procedure to replace the proximal end of a patient's tibia includes a tibial tray and a polymer impaction handle. The tibial tray includes a platform with a fixation member extending downwardly from an inferior surface thereof. The platform has a posterior buttress extending along a posterior section of a perimeter of the platform and extending upwardly from a superior surface of the platform and an anterior buttress extending along an anterior section of the perimeter of the platform and extending upwardly from the superior surface of the platform. The platform also has an elongated threaded bore having a superior end that opens into a superior surface of the posterior buttress. The polymer impaction handle includes an impact plate defining a proximal end of the impaction handle and an impact head defining a distal end of the impaction handle. The impact head has an impact surface that includes a posterior recess that is sized and shaped to receive the posterior buttress of the tibial tray and an anterior recess that is sized and shaped to receive the anterior buttress of the tibial tray. The impaction handle also includes an elongated body extending between the impact plate and the impact head and a locking mechanism positioned in the impact head. The locking mechanism includes a thumbwheel that is rotatable relative to the impact head and a locking shaft secured to a distal surface of the thumbwheel such that a threaded distal end of the locking shaft extends outwardly through an opening formed in the impact surface of the impact head and into the threaded bore of the tibial tray.

In an embodiment, rotation of the thumbwheel in a first direction causes the threaded distal end of the locking shaft to threadingly engage the threaded bore of the tibial tray when the threaded distal end of the locking shaft is positioned therein. In such an embodiment, rotation of the thumbwheel in a direction opposite the first direction causes the threaded distal end of the locking shaft to threadingly disengage the threaded bore of the tibial tray so as to allow the locking shaft to be removed therefrom.

In another embodiment, the locking mechanism further includes a spring with such a spring asserting a spring bias on the distal surface of the thumbwheel so as to urge the thumbwheel toward the impact plate.

In an embodiment, the impact plate, the impact head, and the elongated shaft of the impaction handle are constructed of polyphenylsulfone.

According to another aspect, an orthopaedic system for use in a surgical procedure to replace the proximal end of a patient's tibia includes a tibial tray, a polymer impaction insert, and a metallic impaction handle removably secured to the impaction insert. The tibial tray includes a platform with a fixation member extending downwardly from an inferior surface thereof. The platform has a posterior buttress extending along a posterior section of a perimeter of the platform and extending upwardly from a superior surface of the platform and an anterior buttress extending along an anterior section of the perimeter of the platform and extending upwardly from the superior surface of the platform. The platform also has an elongated threaded bore having a superior end that opens into a superior surface of the posterior buttress. The polymer impaction insert includes a proximal surface and a distal surface that defines an impact surface. The impact surface of the impaction insert includes a posterior recess that is sized and shaped to receive the posterior buttress of the tibial tray and an anterior recess that is sized and shaped to receive the anterior buttress of the tibial tray. The impaction insert also includes a central bore extending between the proximal surface and the distal surface of the impaction insert. The metallic impaction handle has an impact plate defining a proximal end of the impaction handle and an impact head defining a distal end of the impaction handle. The impact head includes an impact surface that is sized and shaped to be positioned on the proximal surface of the impaction insert and an elongated body extending between the impact plate and the impact head. The impaction head also includes a locking mechanism positioned in the impact head. The locking mechanism includes a thumbwheel that is rotatable relative to the impact head and a locking shaft secured to a distal surface of the thumbwheel such that a threaded distal end of the locking shaft extends outwardly through an opening formed in the impact surface of the impact head, through the central bore of the impaction insert, and into the threaded bore of the tibial tray.

In an embodiment, rotation of the thumbwheel in a first direction causes the threaded distal end of the locking shaft to threadingly engage the threaded bore of the tibial tray when the threaded distal end of the locking shaft is positioned therein. In such an embodiment, rotation of the thumbwheel in a direction opposite the first direction causes the threaded distal end of the locking shaft to threadingly disengage the threaded bore of the tibial tray so as to allow the locking shaft to be removed therefrom.

In another embodiment, the locking mechanism further includes a spring with such a spring asserting a spring bias on the distal surface of the thumbwheel so as to urge the thumbwheel toward the impact plate.

In an embodiment, the impaction insert is constructed of polyphenylsulfone, and the impaction handle is constructed of stainless steel.

In another embodiment, the impact surface of the impact head of the impaction handle has a medial buttress and a lateral buttress formed therein. In such an embodiment, the proximal surface of the impaction insert includes a medial recess that is sized and shaped to receive the medial buttress of the impact surface of the impact head of the impaction handle and a lateral recess that is sized and shaped to receive the lateral buttress of the impact surface of the impact head of the impaction handle.

In an embodiment, the locking shaft of the impaction handle has an annular ring formed thereon and the impaction insert has an annular groove defined therein. The annular groove is coaxial with, and opens into, the central bore of the impaction insert. The annular ring of the locking shaft is captured in the annular groove so as to secure the impaction insert to the impaction handle.

### BRIEF DESCRIPTION OF THE DRAWINGS

The detailed description particularly refers to the following figures, in which:

FIG. 1 is an anterior perspective view of an impaction handle for use in implanting a tibial tray of an orthopaedic knee prosthesis;

FIG. 2 is a posterior perspective view of the impaction handle of FIG. 1;

FIG. 3 is an elevation view of the impact head of the impaction handle of FIG. 1;

FIG. 4 is an enlarged fragmentary view of the impaction handle of FIG. 1 showing the handle's impact head secured to a tibial tray;

FIG. 5 is an anterior elevation view of another embodiment of impaction handle and associated impaction insert for use in implanting a tibial tray of an orthopaedic knee prosthesis;

FIG. 6 is a posterior perspective view of the impaction handle and impaction insert of FIG. 5;

FIG. 7 is a proximal perspective view of the impaction insert of FIG. 5;

FIG. 8 is an elevation view of the impact surface of the impaction insert of FIG. 5;

FIG. 9 is a cross-sectional view of the impaction insert taken along the line 9-9 of FIG. 7, as viewed in the direction of the arrows;

FIG. 10 is an enlarged, fragmentary exploded view of the impaction handle and impaction insert of FIG. 5;

FIG. 11 is an enlarged fragmentary view of the impaction handle and impaction insert of FIG. 5 showing the handle and the insert secured to a tibial tray;

FIGS. 12 and 13 are views similar to FIG. 11, but showing the impaction handle embodied with a release spring; and

FIG. 14 is a perspective view of a tibial tray of an orthopaedic knee prosthesis.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention.

Terms representing anatomical references, such as anterior, posterior, medial, lateral, superior, inferior, etcetera, may be used throughout this disclosure in reference to both the orthopaedic implants described herein and a patient's natural anatomy. Such terms have well-understood meanings in both the study of anatomy and the field of orthopaedics. Use of such anatomical reference terms in the specification and claims is intended to be consistent with their well-understood meanings unless noted otherwise.

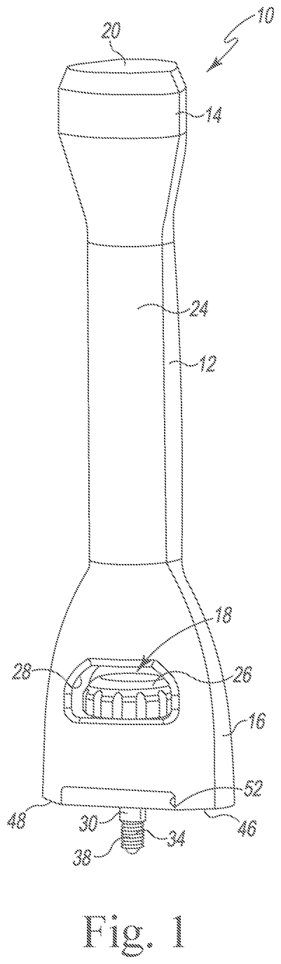

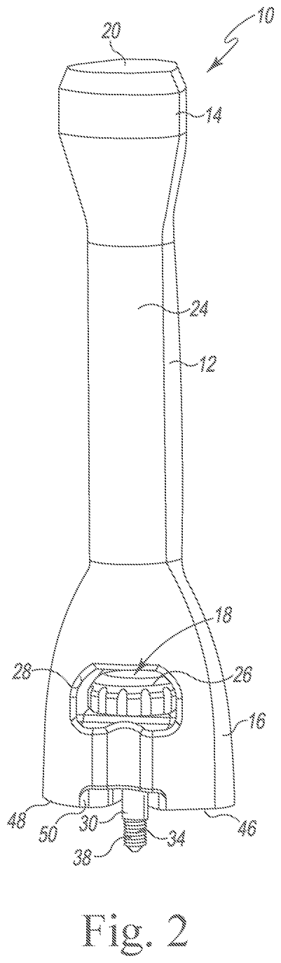

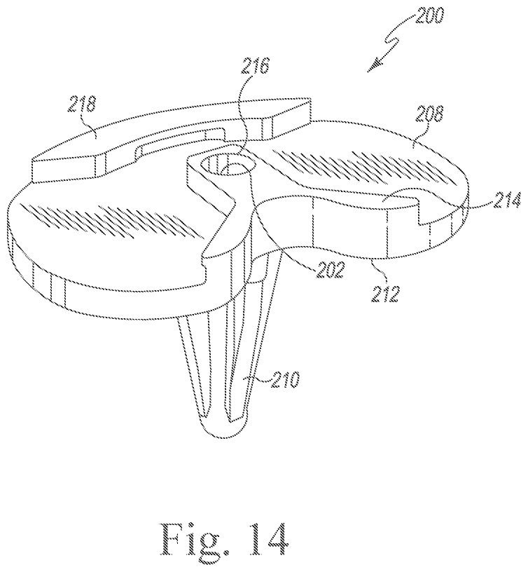

Referring now to FIGS. 1-4, there is shown an orthopaedic surgical instrument—illustratively embodied as an impaction handle 10—for implanting a fixed-bearing tibial tray 200 (see FIG. 14) of a knee prosthesis into a surgically-prepared proximal end of a patient's tibia during an orthopaedic surgical procedure. The impaction handle 10 includes an elongated shaft 12 having an impact plate 14 on its proximal end and an impact head 16 on its distal end. A locking mechanism 18 is positioned in the impact head 14. The locking mechanism is operable to lock the impaction handle 10 to the tibial tray 200 of a knee prosthesis during implantation of the tray.

In the illustrative embodiment, the impaction handle 10 is formed from a polymer material such as, for example, polyphenylsulfone (one suitable polyphenylsulfone is sold under the trademark Radel® and is commercially available from Solvay America, Incorporated of Houston, Texas). In particular, the elongated shaft 12, the impact plate 14, and the impact head 16 form a single monolithic polymer component. The impaction handle 10 may be formed by conventional molding techniques, or alternatively, by the use of 3-D printing technology. In the case of 3-D printing, the impaction handle 10 is formed in a layer-by-layer fashion.

In the exemplary embodiment described herein, the impact plate 14 of the impaction handle 10 includes a rounded polymer strike surface 20 formed in the proximal end of the impact plate 14. In use, the surgeon holds the impaction handle 10 via a grip 24 and strikes the strike surface 20 with a surgical mallet, sledge, or other impaction tool to drive the tibial tray 200 into the surgically-prepared proximal end of the patient's tibia. The impact plate 14 may also be embodied with one or more flanges extending radially outwardly therefrom (not shown). Such flanges serve to protect the surgeon's hand on the grip 24 during impaction. Moreover, such flanges can be impacted from their underside surface if the impaction handle 10 is used to extract a tibial tray 200.

The locking mechanism 18 of the impaction handle 10 is configured to lock the impaction handle 10 to the tibial tray 200 during installation of the same in the patient's surgically-prepared proximal tibia. Although other mechanisms may be used, in the exemplary embodiment described herein, the locking mechanism 18 includes a thumbwheel 26 that is captured within the handle's impact head 16. The thumbwheel 26 is rotatable relative to the impact head 16 and is accessible to a user through an opening 28 formed therein. The locking mechanism 18 also includes a locking shaft 30. A proximal end of the locking shaft 30 is integrally formed with, or otherwise secured to, a distal surface 32 of the thumbwheel 26. The opposite distal end 34 of the locking shaft 30 is threaded and extends out of the impact head 16. Specifically, as can be seen in FIGS. 1-3, the distal end 34 of the locking shaft 30 extends outwardly through an opening 36 formed in the impact surface defined by the distal surface of the impact head 16. As can also be seen in FIGS. 1-3, the distal end 34 of the locking shaft 30 has a set of locking threads 38 defined therein. The locking threads 38 are configured to threadingly engage a threaded bore 202 of the tibial tray 200 so as to secure the tray 200 to the impaction handle 10. Specifically, the threads 38 are sized to be received into the complimentary threads 204 of the tray's threaded bore 202. When a surgeon or other user rotates the thumbwheel 26, the locking shaft's threads 38 are likewise rotated. Rotation in one direction (e.g., clockwise) may be used to tighten, and hence secure, the impaction handle 10 to the tibial tray 200, with rotation in the opposite direction (e.g., counterclockwise) being used to loosen, and hence, uncouple the impaction handle 10 from the tibial tray 200.

As shown in FIGS. 1, 2, and 4, the thumbwheel 26 has a number of blind slots 42 formed in its peripheral sidewall 44. Such a textured surface provides for ease of gripping the thumbwheel 26 during rotation thereof. Moreover, the surfaces of the sidewall 44 defining the slots 42 provide impact surfaces for a tool, such as a flat-bladed screwdriver, to facilitate removal of the impaction handle 10 from a tibial tray 200 in the event the handle is cross threaded to the tray.

As shown in FIG. 14, the tibial tray 200 includes a platform 208 having a fixation member, such as an elongated stem 210, extending away from its inferior surface 212. The elongated tibial stem 210 is configured to be implanted into a surgically prepared end of a patient's tibia (not shown). A generally Y-shaped posterior buttress 214 extends upwardly from the superior surface 206 of the tibial tray 200. In the illustrative embodiment described herein, the posterior buttress 214 has a pair of arms extending along a posterior section of the perimeter of tibial tray's platform 208, along with a third arm extending anteriorly away from the intersection of such a pair of arms (i.e., in a direction toward the center of the platform 208). As can also be seen in FIG. 14, a superior end 216 of the tray's elongated threaded bore 202 opens into the superior surface of the posterior buttress 214. As further shown in FIG. 14, an arcuate-shaped anterior buttress 218 extends upwardly from the superior surface 206 of the tibial tray 200. The anterior buttress 218 extends along an anterior section of the perimeter of tibial tray's platform 208.

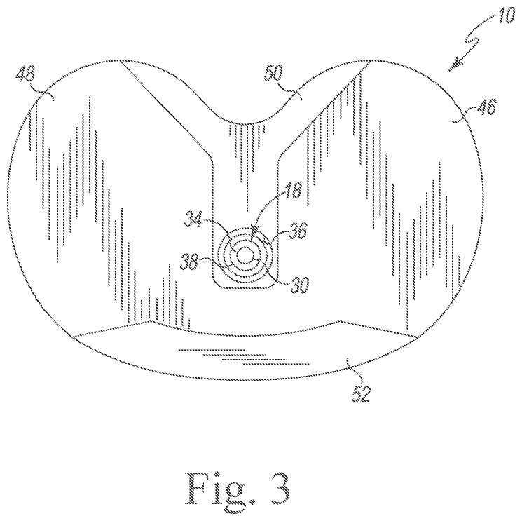

As can be seen in FIGS. 1-3, the distal end 46 of the impact head 16 defines an impact surface 48 that is sized and shaped to be positioned on the superior surface 206 of the tibial tray 200 when the impaction handle 10 is used to impact the tibial tray 200. Specifically, the impact surface 48 includes a generally Y-shaped posterior recess 50 that is sized and shaped to receive the posterior buttress 214 of the tibial tray 200 and an arcuate-shaped anterior recess 52 that is sized and shaped to receive the tray's anterior buttress 218. As can also be seen in FIG. 3, the opening 36 through which the threaded distal end 34 of the locking shaft 30 extends is positioned in the posterior recess 50 so as to align with the tray's threaded bore 202 when the impaction handle's impact head 16 is positioned on the superior surface 206 of the tibial tray 200. It should be appreciated that the closely conforming configuration of the impact surface 16 relative to the features of the tibial tray 200 allows for a relatively high impact load transfer efficiency (i.e., impact load transfer efficiency=output force/input force) of the handle without the transfer of significant forces through the threaded locking shaft 30.

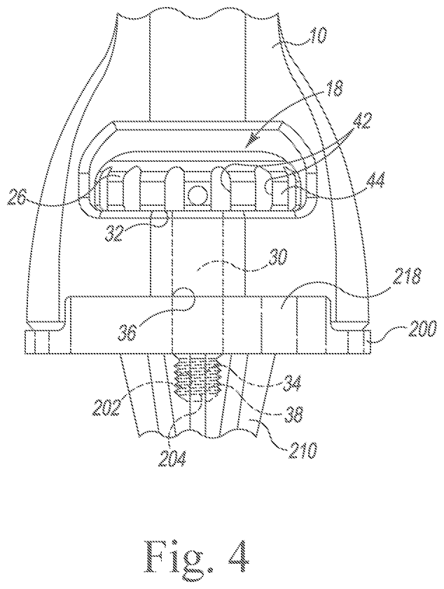

In use, the impaction handle 10 may be utilized by a surgeon to implant the tibial tray 200 into the surgically-prepared proximal end of a patient's tibia. To do so, the surgeon first secures the tibial tray 200 to the handle 10. Specifically, as shown in FIG. 4, the impact head 16 of the impaction handle 10 is placed on the superior surface 206 of the tibial tray 200 such that the tray's posterior buttress 214 is positioned in the impact head's posterior recess 50 thereby also positioning the tray's anterior buttress 218 in the impact head's anterior recess 52. By doing so, the threaded distal end 34 of the locking shaft 30 is initially advanced into the superior end 216 of the tray's elongated threaded bore 202. Thereafter, the surgeon rotates the thumbwheel 26 in the direction (e.g., clockwise) to cause the threaded distal end 34 of the locking shaft 30 to threadingly engage the tray's threaded bore 202 thereby securing the impaction handle 10 to the tibial tray 200.

Thereafter, the surgeon uses the impaction handle 10 to position the tibial tray 200 such that its inferior bone-engaging surface 212, along with its stem 210, is positioned relative to the patient's surgically-prepared proximal tibia in a desired orientation. Once the tibial tray 200 is positioned in such a manner, the surgeon strikes the impact plate 14 of the impaction handle 10 with a surgical mallet, sledge, or other impaction tool to drive the tibial tray 200 into the bone tissue until the tibial tray 200 is fully seated on the patient's surgically-prepared proximal tibia.

The surgeon then releases the tibial tray 200 from the impaction handle 10. To do so, the surgeon rotates the thumbwheel 26 in the opposite direction (e.g., counterclockwise) thereby threadingly disengaging the threaded distal end 34 of the locking shaft 30 from the tray's threaded bore 202 thus releasing the impaction handle from the tibial tray 200.

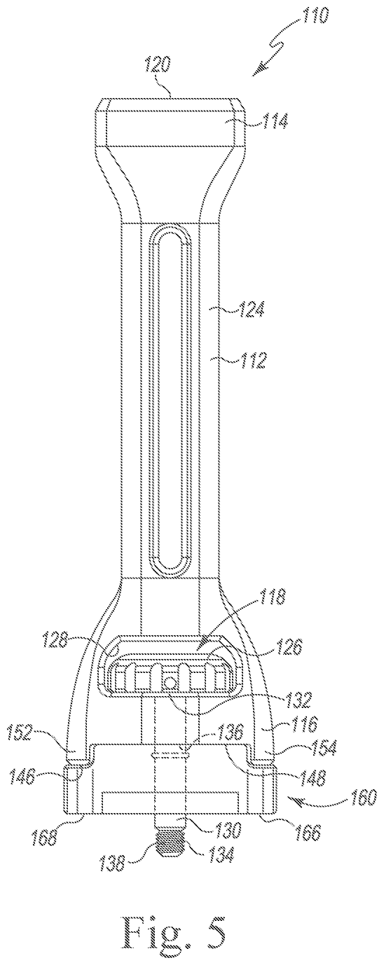

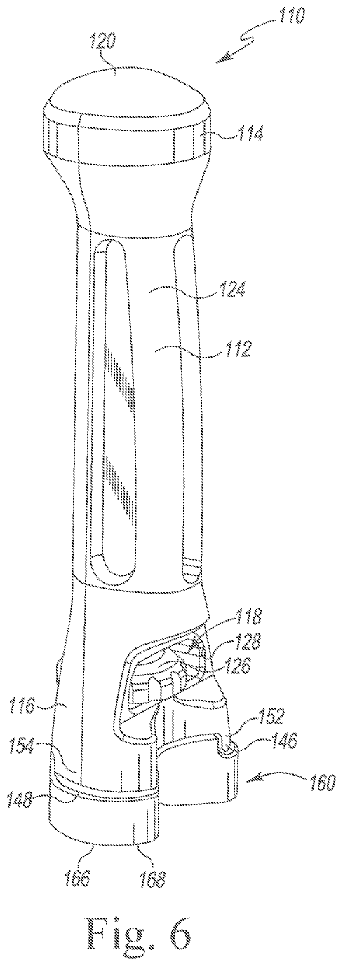

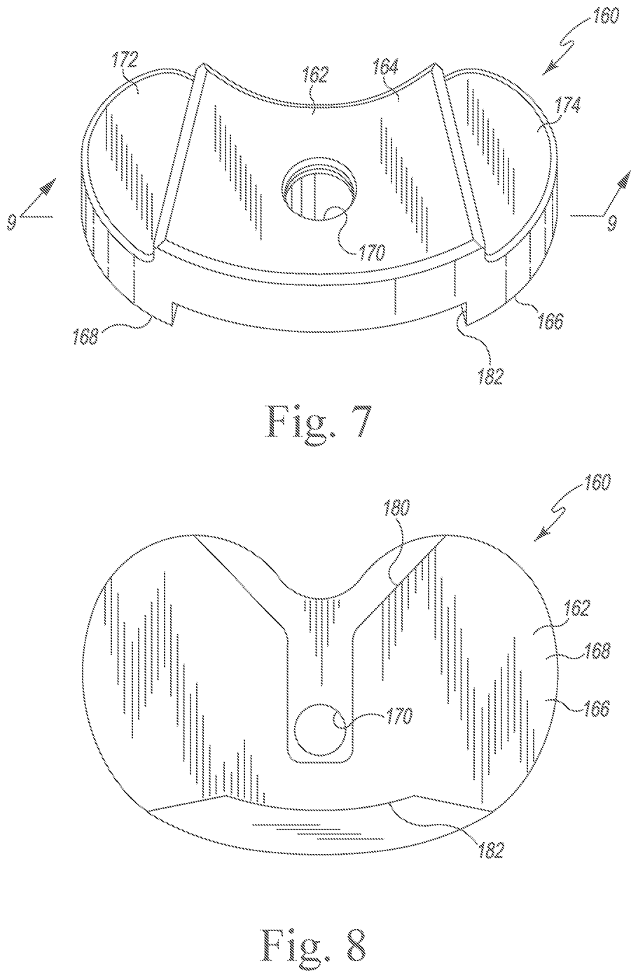

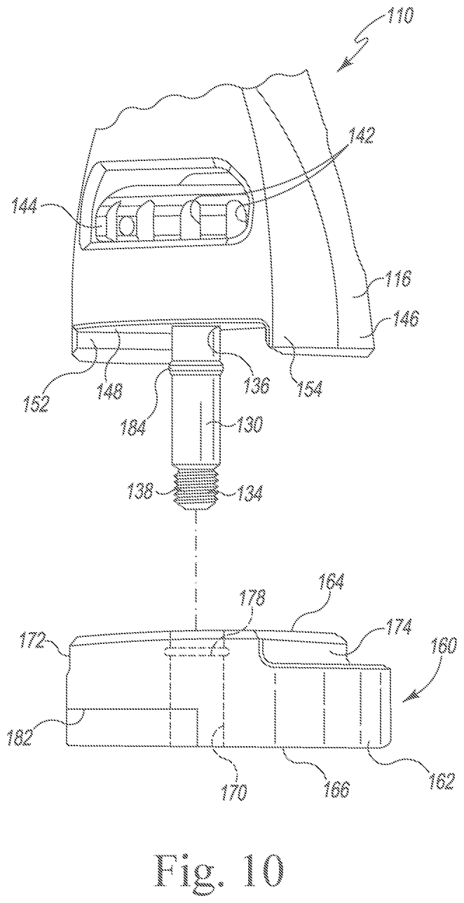

Referring now to FIGS. 5-11, there is shown another embodiment of an orthopaedic surgical instrument—illustratively embodied as an impaction handle 110 and an impaction insert 160—for implanting the fixed-bearing tibial tray 200 of FIG. 14. The assembly of the impaction handle 110 and the impaction insert 160 is similar to the impaction handle 10 of FIGS. 1-4 and includes many similar features. As will be discussed below in greater detail, the two-piece design of the instrument of FIGS. 5-11 allows for certain features of the instrument to be fabricated with differing materials.

The impaction handle 110 includes an elongated shaft 112 having an impact plate 114 on its proximal end and an impact head 116 on its distal end. A locking mechanism 118 is positioned in the impact head 14. The locking mechanism is operable to lock the impaction handle 110 to the tibial tray 200 during implantation of the tray.

In the illustrative embodiment, the impaction handle 110 is formed from a metallic material such as, for example, stainless steel. In particular, the elongated shaft 112, the impact plate 114, and the impact head 116 form a single monolithic metallic component. The impaction handle 110 may be formed by conventional machining techniques, or alternatively, by the use of 3-D printing technology. In the case of 3-D printing, the impaction handle 110 is formed in a layer-by-layer fashion.

In the exemplary embodiment described herein, the impact plate 114 of the impaction handle 110 includes a rounded metal strike surface 120 formed in the proximal end of the impact plate 114. In use, the surgeon holds the impaction handle 110 via a grip 124 and strikes the strike surface 120 with a surgical mallet, sledge, or other impaction tool to drive the tibial tray 200 into the surgically-prepared proximal end of the patient's tibia. The impact plate 114 may also be embodied with one or more flanges extending radially outwardly therefrom (not shown). Such flanges serve to protect the surgeon's hand on the grip 124 during impaction. Moreover, such flanges can be impacted from their underside surface if the impaction handle 110 is used to extract a tibial tray 200.

The locking mechanism 118 of the impaction handle 110 is similar to the locking mechanism 18 of the impaction handle 10 of FIGS. 1-4 and is thus configured to lock the impaction handle 10 to the tibial tray 200 during installation of the same in the patient's surgically-prepared proximal tibia. In addition, as will be discussed below, the locking mechanism 18 is also configured to selectively secure the impaction insert 160 to the impaction handle 110. Although other mechanisms may be used, in the exemplary embodiment described herein, the locking mechanism 118 includes a thumbwheel 126 that is captured within the handle's impact head 116. The thumbwheel 126 is similar to the thumbwheel 26 of FIGS. 1-4 and, as a result, is rotatable relative to the impact head 116 and is accessible to a user through an opening 128 formed therein. The locking mechanism 118 also includes a locking shaft 130. A proximal end of the locking shaft 130 is integrally formed with, or otherwise secured to, a distal surface 132 of the thumbwheel 126. The opposite distal end 134 of the locking shaft 130 is threaded and extends out of the impact head 116. Specifically, as can be seen in FIGS. 5, 10, and 11, the distal end 134 of the locking shaft 130 extends outwardly through an opening 136 formed in the impact surface defined by the distal surface of the impact head 116. Moreover, the locking shaft 130 is longer than the locking shaft 30 of FIGS. 1-4 so as to allow the shaft 130 to also pass through the impaction insert 160. As can also be seen in FIGS. 5, 10, and 11, the distal end 134 of the locking shaft 130 has a set of locking threads 138 defined therein. The locking threads 138 are identical to the locking threads 38 of the impaction handle 10 and are thus configured to threadingly engage a threaded bore 202 of the tibial tray 200 so as to secure the tray 200 to the impaction handle 110. Specifically, the threads 138 are sized to be received into the complimentary threads 204 of the tray's threaded bore 202. When a surgeon or other user rotates the thumbwheel 216, the locking shaft's threads 138 are likewise rotated. Rotation in one direction (e.g., clockwise) may be used to tighten, and hence secure, the impaction handle 110 to the tibial tray 200, with rotation in the opposite direction (e.g., counterclockwise) being used to loosen, and hence, uncouple the impaction handle 110 from the tibial tray 200.

As shown in FIGS. 5, 6, 10, and 11, the thumbwheel 126 has a number of blind slots 142 formed in its peripheral sidewall 144. Such a textured surface provides for ease of gripping the thumbwheel 126 during rotation thereof. Moreover, the surfaces of the sidewall 144 defining the slots 142 provide impact surfaces for a tool, such as a flat-bladed screwdriver, to facilitate removal of the impaction handle 110 from a tibial tray 200 in the event the handle is cross threaded to the tray.

The impaction insert 160 has a polymer body 162 that has a similar size and profile shape as both the impact head 116 of the impaction handle 110 and the platform 208 of the tibial tray 200. The impaction insert 160 includes a proximal surface 164 that is sized and shaped to correspond with the impact surface of the impaction handle 110. Specifically, as can be seen in FIGS. 5, 6, 10, and 11, the distal end 146 of the handle's impact head 116 defines an impact surface 148 that has a medial buttress 152 and a lateral buttress 154 formed therein. The proximal surface 164 of the impaction insert 160 has formed therein both a medial recess 172 that is sized and shaped to receive the medial buttress 152 of the impact surface 148 of the impact head 116 of the impaction handle 110, and a lateral recess 174 that is sized and shaped to receive the lateral buttress 154 of the impact surface 148 of the impact head 116 of the impaction handle 110.

The opposite distal surface 166 of the impaction insert 160 defines an impact surface 168 that is sized and shaped to be positioned on the superior surface 206 of the tibial tray 200 when the impaction handle 110 and the impaction insert 160 are used to impact the tibial tray 200. Specifically, the impact surface 168 includes a generally Y-shaped posterior recess 180 that is sized and shaped to receive the posterior buttress 214 of the tibial tray 200 and an arcuate-shaped anterior recess 182 that is sized and shaped to receive the tray's anterior buttress 218.

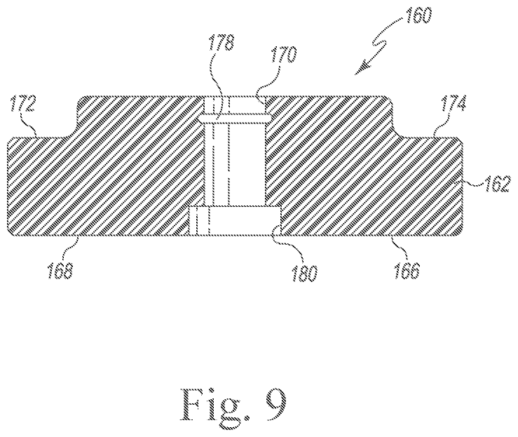

The impaction insert 160 also includes a smooth central bore 170 extending between its proximal surface 164 and its distal surface 166. As can be seen in FIGS. 8 and 9, the central bore 170 opens into the posterior recess 180. In such a way, the tray's threaded bore 202, the central bore 170 of the impaction insert 160, and the opening 136 in the handle's impact surface 148 through which the threaded distal end 134 of the locking shaft 130 extends are each aligned with one another when the impaction insert 160 is positioned on the superior surface 206 of the tibial tray 200. It should be appreciated that the closely conforming configuration of the insert's impact surface 168 relative to the features of the tibial tray 200 allows for a relatively high impact load transfer efficiency (i.e., impact load transfer efficiency=output force/input force) of the insert without the transfer of significant forces through the threaded locking shaft 130.

As shown in FIGS. 5, 10, and 11, the locking shaft 130 of the impaction handle 110 has an annular ring 184 formed thereon. The annular ring 184 is utilized to removably secure the impaction insert 160 to the impaction handle 110. In particular, as shown in FIGS. 5 and 9-11, the impaction insert 160 has an annular groove 178 defined therein. The annular groove 178 is coaxial with, and opens into, the central bore 170 of the impaction insert 160. The annular ring 184 of the locking shaft 130 may be captured in the annular groove 178 so as to secure the impaction insert 160 to the impaction handle 110. However, a removal force (e.g., pulling force) of sufficient magnitude may be applied to the impaction insert 160 such that the annular ring 184 is freed from the annular groove 178 thereby allowing the impaction insert 160 to be pulled off of, or otherwise removed from, the locking shaft 130.

Although the impaction handle 110 is metallic, the impaction insert 160 is embodied as a single monolithic component formed from a polymer material such as, for example, polyphenylsulfone (e.g., Radel®). The impaction insert 160 may be formed by conventional molding techniques, or alternatively, by the use of 3-D printing technology. In the case of 3-D printing, the impaction insert 160 is formed in a layer-by-layer fashion. The use of differing materials allows the portion of the instrument that is repeatedly impacted by the surgeon (i.e., the impaction handle 110) to be constructed of a fairly hard material (i.e., metal) while also allowing the portion of the instrument that interfaces with the tibial tray 200 (i.e., the insert component 160) to be constructed with a softer material (i.e., polymer).

In use, the impaction handle 110 and the impaction insert 160 may be utilized by a surgeon to implant the tibial tray 200 into the surgically-prepared proximal end of a patient's tibia. To do so, the surgeon first secures the impaction insert 160 to the impaction handle 110. To do so, the surgeon inserts the threaded distal end 134 of the locking shaft 130 into the central bore 170 of the impaction insert 160 such that the annular ring 184 of the locking shaft 130 is captured in the insert's annular groove 178.

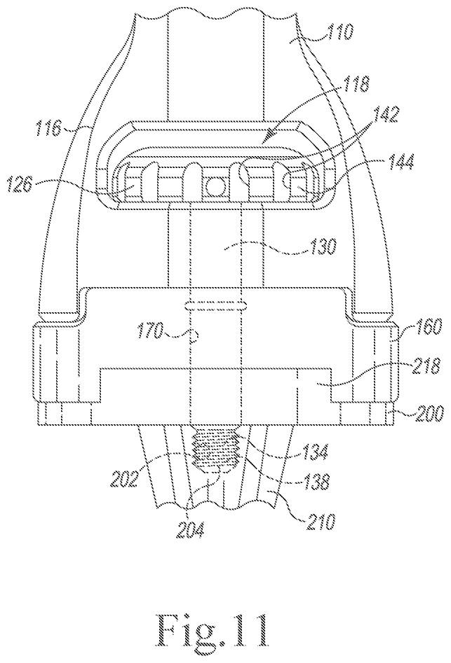

The surgeon then secures the tibial tray 200 to the assembled instrument. Specifically, as shown in FIG. 11, the impact surface 168 of the impaction insert 160 is placed on the superior surface 206 of the tibial tray 200 such that the tray's posterior buttress 214 is positioned in the insert's posterior recess 180 thereby also positioning the tray's anterior buttress 218 in the impact head's anterior recess 182. By doing so, the threaded distal end 134 of the locking shaft 130 is initially advanced into the superior end 216 of the tray's elongated threaded bore 202. Thereafter, the surgeon rotates the thumbwheel 126 in the direction (e.g., clockwise) to cause the threaded distal end 134 of the locking shaft 130 to threadingly engage the tray's threaded bore 202 thereby securing the impaction handle 110 and the impaction insert 160 to the tibial tray 200.

Thereafter, the surgeon uses the impaction handle 110 to position the tibial tray 200 such that its inferior bone-engaging surface 212, along with its stem 210, is positioned relative to the patient's surgically-prepared proximal tibia in a desired orientation. Once the tibial tray 200 is positioned in such a manner, the surgeon strikes the impact plate 114 of the impaction handle 110 with a surgical mallet, sledge, or other impaction tool to drive the tibial tray 200 into the bone tissue until the tibial tray 200 is fully seated on the patient's surgically-prepared proximal tibia.

The surgeon then releases the tibial tray 200 from the impaction handle 110. To do so, the surgeon rotates the thumbwheel 126 in the opposite direction (e.g., counterclockwise) thereby threadingly disengaging the threaded distal end 134 of the locking shaft 130 from the tray's threaded bore 202 thus releasing the impaction handle 110 and the impaction insert 160 from the tibial tray 200. If the so desired, the surgeon may also remove the impaction insert 160 from the impaction handle by pulling on the insert 160 with sufficient force to allow the shaft's annular ring 184 to be freed from the annular groove 178 thereby allowing the impaction insert 160 to be pulled off the locking shaft 130.

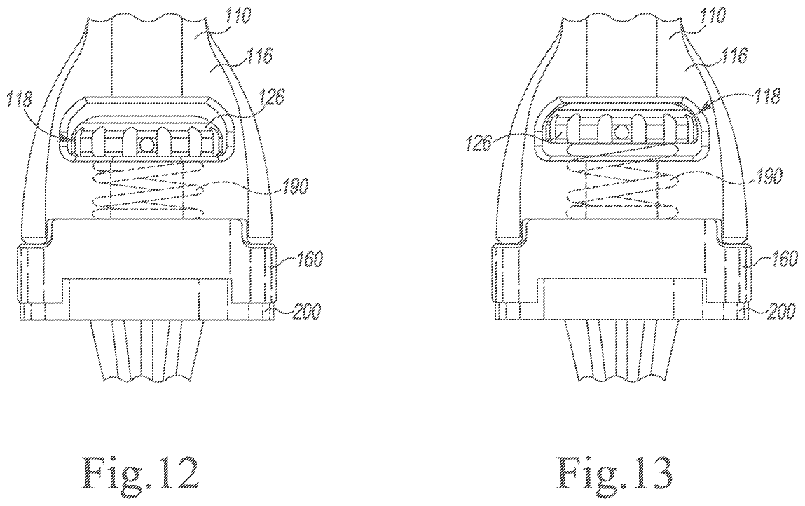

As shown in FIGS. 12 and 13, the locking mechanism 118 of the impaction handle may be embodied with a release spring 190. The release spring 190 is positioned around the locking shaft 130 and asserts a spring bias on the distal surface 132 of the thumbwheel 126 that urges the thumbwheel 126, and hence the locking shaft 130, proximally (i.e., in a direction toward the handle's impact plate 114). As such, the spring 190 facilitates release of the instrument from the tibial tray 200 after impaction. In particular, as the surgeon rotates the thumbwheel 126 to loosen the threaded distal end 134 of the locking shaft 130 from the tray's threaded bore 202, the spring 190 causes the thumbwheel 126 to “pop” upwardly once the threaded locking shaft 130 has disengaged the tray's threaded bore 202 thereby providing an indication to the surgeon that the instrument is released from the implanted tray 200.

It should be appreciated that a slight downward force may be applied to the thumbwheel 126 to overcome the spring bias of the release spring 190 during installation of the instrument onto the tibial tray 200. Specifically, during assembly, the surgeon may apply a downward force on the thumbwheel 126 to urge the leading threads of the threaded locking shaft 130 into engagement with the threads of the tray's threaded bore 202.

Although use of the release spring 190 is shown in FIGS. 12 and 13 in regard to the impaction handle 110, it should be appreciated that the impaction handle 10 of FIGS. 1-4 could also be embodied with the release spring in a similar manner.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

For example, it should be appreciated that the concepts described herein may be utilized in the design of impaction handles for use in implanting other types of orthopaedic implants such as hip implants, shoulder implants, or other components (e.g., femoral) of a knee prosthesis.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. An orthopaedic surgical instrument for use during a surgical procedure to implant a tibial tray into a surgically-prepared proximal end of a tibia, comprising:
an impaction handle, comprising:
an impact plate defining a proximal end of the impaction handle,
an impact head defining a distal end of the impaction handle, the impact head having an impact surface that is sized and shaped to be positioned on a superior surface of the tibial tray when the impaction handle is used to impact the tibial tray,
an elongated body extending between the impact plate and the impact head, and
a locking mechanism positioned in the impact head, the locking mechanism comprising (i) a thumbwheel that is rotatable relative to the impact head, and (ii) a locking shaft secured to a distal surface of the thumbwheel such that a threaded distal end of the locking shaft extends outwardly through an opening formed in the impact surface of the impact head, wherein (i) the threaded distal end of the locking shaft is configured to be threadingly received into a threaded bore formed in the tibial tray, (ii) rotation of the thumbwheel causes rotation of the locking shaft so as to cause its threaded distal end to selectively threadingly engage the threaded bore of the tibial tray when the threaded distal end of the locking shaft is positioned therein,
wherein the impact plate, the impact head, and the elongated shaft of the impaction handle collectively define a single monolithic polymer component.

2. The orthopaedic surgical instrument of claim 1, wherein:
rotation of the thumbwheel in a first direction causes the threaded distal end of the locking shaft to threadingly engage the threaded bore of the tibial tray when the threaded distal end of the locking shaft is positioned therein, and
rotation of the thumbwheel in a direction opposite the first direction causes the threaded distal end of the locking shaft to threadingly disengage the threaded bore of the tibial tray so as to allow the locking shaft to be removed therefrom.

3. The orthopaedic surgical instrument of claim 1, wherein:
the locking mechanism further comprises a spring, and
the spring asserts a spring bias on the distal surface of the thumbwheel so as to urge the thumbwheel toward the impact plate.

4. The orthopaedic surgical instrument of claim 1, wherein the impact plate, the impact head, and the elongated shaft of the impaction handle are constructed of polyphenylsulfone.

5. An orthopaedic surgical instrument for use during a surgical procedure to implant a tibial tray into a surgically-prepared proximal end of a tibia, comprising:
a polymer impaction insert having (i) a proximal surface, (ii) a distal surface that defines an impact surface that is sized and shaped to be positioned on a superior surface of the tibial tray when the impaction insert is used to impact the tibial tray, and (iii) a central bore extending between the proximal surface and the distal surface of the impaction insert, and
a metallic impaction handle removably secured to the impaction insert, the impaction handle having (i) an impact plate defining a proximal end of the impaction handle, (ii) an impact head defining a distal end of the impaction handle, the impact head having an impact surface that is sized and shaped to be positioned on the proximal surface of the impaction insert, (iii) an elongated body extending between the impact plate and the impact head, and (iv) a locking mechanism positioned in the impact head, the locking mechanism comprising (a) a thumbwheel that is rotatable relative to the impact head, and (b) a locking shaft secured to a distal surface of the thumbwheel such that a threaded distal end of the locking shaft extends outwardly through an opening formed in the impact surface of the impact head and through the central bore of the impaction insert,
wherein (i) the threaded distal end of the locking shaft is configured to be threadingly received into a threaded bore formed in the tibial tray, (ii) rotation of the thumbwheel causes rotation of the locking shaft so as to cause its threaded distal end to selectively threadingly engage the threaded bore of the tibial tray when the threaded distal end of the locking shaft is positioned therein.

6. The orthopaedic surgical instrument of claim 5, wherein:
rotation of the thumbwheel in a first direction causes the threaded distal end of the locking shaft to threadingly engage the threaded bore of the tibial tray when the threaded distal end of the locking shaft is positioned therein, and
rotation of the thumbwheel in a direction opposite the first direction causes the threaded distal end of the locking shaft to threadingly disengage the threaded bore of the tibial tray so as to allow the locking shaft to be removed therefrom.

7. The orthopaedic surgical instrument of claim 5, wherein:
the locking mechanism further comprises a spring, and
the spring asserts a spring bias on the distal surface of the thumbwheel so as to urge the thumbwheel toward the impact plate.

8. The orthopaedic surgical instrument of claim 5, wherein:
the impaction insert is constructed of polyphenylsulfone, and
the impaction handle is constructed of stainless steel.

9. The orthopaedic surgical instrument of claim 5, wherein:
the impact surface of the impact head of the impaction handle has a medial buttress and a lateral buttress formed therein, and
the proximal surface of the impaction insert comprises (i) a medial recess that is sized and shaped to receive the medial buttress of the impact surface of the impact head of the impaction handle, and (ii) a lateral recess that is sized and shaped to receive the lateral buttress of the impact surface of the impact head of the impaction handle.

10. The orthopaedic surgical instrument of claim 5, wherein:
the locking shaft of the impaction handle has an annular ring formed thereon,
the impaction insert has an annular groove defined therein, the annular groove is coaxial with, and opens into, the central bore of the impaction insert, and
the annular ring of the locking shaft is captured in the annular groove so as to secure the impaction insert to the impaction handle.

11. An orthopaedic system for use in a surgical procedure to replace the proximal end of a patient's tibia, comprising:
a tibial tray having a platform with a fixation member extending downwardly from an inferior surface thereof, the platform having (i) a posterior buttress extending along a posterior section of a perimeter of the platform and extending upwardly from a superior surface of the platform, (ii) an anterior buttress extending along an anterior section of the perimeter of the platform and extending upwardly from the superior surface of the platform, and (iii) an elongated threaded bore having a superior end that opens into a superior surface of the posterior buttress, and
a polymer impaction handle having (i) an impact plate defining a proximal end of the impaction handle, (ii) an impact head defining a distal end of the impaction handle, the impact head having an impact surface that comprises (a) a posterior recess that is sized and shaped to receive the posterior buttress of the tibial tray, and (b) an anterior recess that is sized and shaped to receive the anterior buttress of the tibial tray, (iii) an elongated body extending between the impact plate and the impact head, and (iv) a locking mechanism positioned in the impact head, the locking mechanism comprising (a) a thumbwheel that is rotatable relative to the impact head, and (b) a locking shaft secured to a distal surface of the thumbwheel such that a threaded distal end of the locking shaft extends outwardly through an opening formed in the impact surface of the impact head and into the threaded bore of the tibial tray.

12. The orthopaedic system of claim 11, wherein:
rotation of the thumbwheel in a first direction causes the threaded distal end of the locking shaft to threadingly engage the threaded bore of the tibial tray when the threaded distal end of the locking shaft is positioned therein, and
rotation of the thumbwheel in a direction opposite the first direction causes the threaded distal end of the locking shaft to threadingly disengage the threaded bore of the tibial tray so as to allow the locking shaft to be removed therefrom.

13. The orthopaedic system of claim 11, wherein:
the locking mechanism further comprises a spring, and
the spring asserts a spring bias on the distal surface of the thumbwheel so as to urge the thumbwheel toward the impact plate.

14. The orthopaedic system of claim 11, wherein the impact plate, the impact head, and the elongated shaft of the impaction handle are constructed of polyphenylsulfone.

15. An orthopaedic system for use in a surgical procedure to replace the proximal end of a patient's tibia, comprising:
a tibial tray having a platform with a fixation member extending downwardly from an inferior surface thereof, the platform having (i) a posterior buttress extending along a posterior section of a perimeter of the platform and extending upwardly from a superior surface of the platform, (ii) an anterior buttress extending along an anterior section of the perimeter of the platform and extending upwardly from the superior surface of the platform, and (iii) an elongated threaded bore having a superior end that opens into a superior surface of the posterior buttress,
a polymer impaction insert having (i) a proximal surface, (ii) a distal surface that defines an impact surface that comprises (a) a posterior recess that is sized and shaped to receive the posterior buttress of the tibial tray, and (b) an anterior recess that is sized and shaped to receive the anterior buttress of the tibial tray, and (iii) a central bore extending between the proximal surface and the distal surface of the impaction insert, and
a metallic impaction handle removably secured to the impaction insert, the impaction handle having (i) an impact plate defining a proximal end of the impaction handle, (ii) an impact head defining a distal end of the impaction handle, the impact head having an impact surface that is sized and shaped to be positioned on the proximal surface of the impaction insert, (iii) an elongated body extending between the impact plate and the impact head, and (iv) a locking mechanism positioned in the impact head, the locking mechanism comprising (a) a thumbwheel that is rotatable relative to the impact head, and (b) a locking shaft secured to a distal surface of the thumbwheel such that a threaded distal end of the locking shaft extends outwardly through an opening formed in the impact surface of the impact head, through the central bore of the impaction insert, and into the threaded bore of the tibial tray.

16. The orthopaedic system of claim 15, wherein:
rotation of the thumbwheel in a first direction causes the threaded distal end of the locking shaft to threadingly engage the threaded bore of the tibial tray when the threaded distal end of the locking shaft is positioned therein, and
rotation of the thumbwheel in a direction opposite the first direction causes the threaded distal end of the locking shaft to threadingly disengage the threaded bore of the tibial tray so as to allow the locking shaft to be removed therefrom.

17. The orthopaedic system of claim 15, wherein:
the locking mechanism further comprises a spring, and
the spring asserts a spring bias on the distal surface of the thumbwheel so as to urge the thumbwheel toward the impact plate.

18. The orthopaedic system of claim 15, wherein:
the impaction insert is constructed of polyphenylsulfone, and
the impaction handle is constructed of stainless steel.

19. The orthopaedic system of claim 15, wherein:
the impact surface of the impact head of the impaction handle has a medial buttress and a lateral buttress formed therein, and
the proximal surface of the impaction insert comprises (i) a medial recess that is sized and shaped to receive the medial buttress of the impact surface of the impact head of the impaction handle, and (ii) a lateral recess that is sized and shaped to receive the lateral buttress of the impact surface of the impact head of the impaction handle.

20. The orthopaedic system of claim 15, wherein:
the locking shaft of the impaction handle has an annular ring formed thereon,
the impaction insert has an annular groove defined therein, the annular groove is coaxial with, and opens into, the central bore of the impaction insert, and
the annular ring of the locking shaft is captured in the annular groove so as to secure the impaction insert to the impaction handle.
